SEQUENCE LISTING (2 sequences)

SEQ ID NO: 1 - DNA, 23 bp, Artificial Sequence
  Forward Primer
cttctaaccgaggtcgaaacgta

SEQ ID NO: 2 - DNA, 22 bp, Artificial Sequence
  Reverse Primer
ttggacaaagcgtctacgctgc

(19) United States
(12) Patent Application Publication
(10) Pub. No.: US 2015/0175948 A1
(43) Pub. Date: Jun. 25, 2015

(54) NUCLEIC ACID AMPLIFICATION REACTOR

(71) Applicant: Kazuki Yamamoto, Kyoto-city (JP)

(72) Inventor: Kazuki Yamamoto, Kyoto-city (JP)

(21) Appl. No.: 14/382,310

(22) PCT Filed: Mar. 2, 2012
(86) PCT No.: PCT/JP2012/001442

§ 371 Date: Aug. 29, 2014

(51) Int. Cl. C12M 1/00; C12M 1/26

(52) U.S. Cl. CPC C12M 23/20; C12M 23/58; C12M 33/00

(57) ABSTRACT

Provided is a nucleic acid amplification reactor that can easily perform a nucleic acid amplification reaction. A nucleic acid amplification reactor 1 includes a reaction chamber 20 to which a thermoplastic hydrogel 50 is applied. The thermoplastic hydrogel 50 contains a DNA polymerase, a set of oligonucleotide primers, a nucleotide, and a gelator.

### TECHNICAL FIELD

This invention relates to nucleic acid amplification reactors.

### BACKGROUND ART

A nucleic acid amplification reaction represented by a PCR method is useful not only as a method for analyzing gene polymorphisms (SNP) of an organism but also as a method for investigating the expression level of a gene introduced into a cell. Furthermore, the nucleic acid amplification reaction is used to find out the gene expression pattern of a cell in a particular state, such as an iPS cell, an ES cell or a cancer cell, and identify a pathogen. In addition, because the nucleic acid amplification reaction enables the amplification of a minute amount of nucleic acid to a visible amount thereof, it is also used as a method for rapidly detecting a microorganism. For example, the amplification of a nucleic acid with which a molecular recognition reagent is labeled, as in an immuno-PCR method, is useful also for detection of a minute amount of microorganism.

Recently, the nucleic acid amplification reaction has also been used to detect a minute amount of RNA using reverse transcriptase. In this case, an approach is taken in which RNA is converted into complementary DNA (cDNA) using reverse transcriptase and cDNA is then amplified by a nucleic acid amplification reaction.

The nucleic acid amplification reaction is carried out using a nucleic acid amplification reaction apparatus, as disclosed in Patent Literature 1, for example.

The nucleic acid amplification reaction apparatus is generally provided with a thermal cycler and other elements. The nucleic acid amplification reaction is performed in a nucleic acid amplification reactor, such as a sample tube, by setting the nucleic acid amplification reactor in the nucleic acid amplification reaction apparatus and controlling the temperature thereof with the thermal cycler.

### CITATION LIST

### Patent Literature

### [PTL 1]

### JP-A-2010-519892

### SUMMARY OF INVENTION

### Technical Problem

A reaction compound including a template DNA, a DNA polymerase, a set of oligonucleotide primers, and a nucleotide is charged into the nucleic acid amplification reactor. The reaction compound to be charged into the nucleic acid amplification reactor has a problem in that since it is composed of many types of components, the preparation of the reaction compound becomes complicated if many target nucleic acids should be concurrently detected or if a large-scale sample set should be analyzed.

A principal object of the present invention is to provide a nucleic acid amplification reactor that can easily perform a nucleic acid amplification reaction.

### Solution to Problem

A nucleic acid amplification reactor of the present invention includes a reaction chamber to which a thermoplastic hydrogel is applied. The thermoplastic hydrogel contains a DNA polymerase, a set of oligonucleotide primers, a nucleotide, and a gelator.

In a particular aspect of the nucleic acid amplification reactor of the present invention, the gel-sol transition temperature of the thermoplastic hydrogel which is a temperature of transition thereof from gel to sol phase is 90 degrees Celsius or below and the sol-gel transition temperature of the thermoplastic hydrogel which is a temperature of transition thereof from sol to gel phase is 55 degrees Celsius or below.

In a particular aspect of the nucleic acid amplification reactor of the present invention, the thermoplastic hydrogel further contains a reporter reagent.

In a particular aspect of the nucleic acid amplification reactor of the present invention, the nucleic acid amplification reactor further includes a thermoplastic hydrogel applied to the reaction chamber and containing a magnesium salt.

In a particular aspect of the nucleic acid amplification reactor of the present invention, the nucleic acid amplification reactor further includes a metallic member provided to extend from an inside wall of the reaction chamber to an outside wall of the nucleic acid amplification reactor.

In a particular aspect of the nucleic acid amplification reactor of the present invention, the nucleic acid amplification reactor includes a plurality of the reaction chambers. The thermoplastic hydrogel applied to each of the plurality of the reaction chambers is of a single type or a combination of types selected from different types of thermoplastic hydrogels different in the type of the set of oligonucleotide primers.

In a particular aspect of the nucleic acid amplification reactor of the present invention, the nucleic acid amplification reactor further includes a microchannel, a weighing part, and a passive valve. The weighing part is connected to the microchannel. The weighing part is provided for each of the reaction chambers. The passive valve connects the weighing part to the reaction chamber.

In a particular aspect of the nucleic acid amplification reactor of the present invention, the nucleic acid amplification reactor includes the seven or more reaction chambers. Each of the seven or more reaction chambers includes the thermoplastic hydrogel applied thereto, the thermoplastic hydrogel containing one or more sets of oligonucleotide primers selected from three or more different sets of oligonucleotide primers. The set of oligonucleotide primers contained in the thermoplastic hydrogel applied to each of the seven or more reaction chambers is selected according to a recurring pseudo-random binary sequence.

### Advantageous Effects of Invention

The present invention can provide a nucleic acid amplification reactor that can easily perform a nucleic acid amplification reaction.

### BRIEF DESCRIPTION OF DRAWINGS

FIG. 1 is a schematic diagram of a nucleic acid amplification reactor of a first embodiment.

FIG. 2 is a schematic cross-sectional view of a substrate of the nucleic acid amplification reactor taken along the line II-II in FIG. 1.

FIG. 3 is a schematic diagram of an array of sets of oligonucleotide primers based on a matrix M defined by a single cycle of a 7-bit M-sequence.

FIG. 4 is a schematic diagram of an array of sets of oligonucleotide primers based on a matrix M defined by three cycles of the 7-bit M-sequence.

FIG. 5 is graphs showing the relation between the amount of DNA fragments and the number of cycles in Example 1 and Reference Example 1.

FIG. 6 is a schematic cross-sectional view of a substrate of a nucleic acid amplification reactor of a second embodiment.

### DESCRIPTION OF EMBODIMENTS

Hereinafter, a description will be given of exemplified preferred embodiments of the present invention. However, the following embodiments are simply illustrative. The present invention is not limited at all to the following embodiments.

Throughout the drawings to which the embodiments and the like refer, elements having substantially the same functions will be referred to by the same reference signs. The drawings to which the embodiments and the like refer are schematically illustrated and, therefore, the dimensional ratios and the like of objects illustrated in the drawings may be different from those of the actual objects. Different drawings may have different dimensional ratios and the like of the objects. Dimensional ratios and the like of specific objects should be determined in consideration of the following descriptions.

### First Embodiment

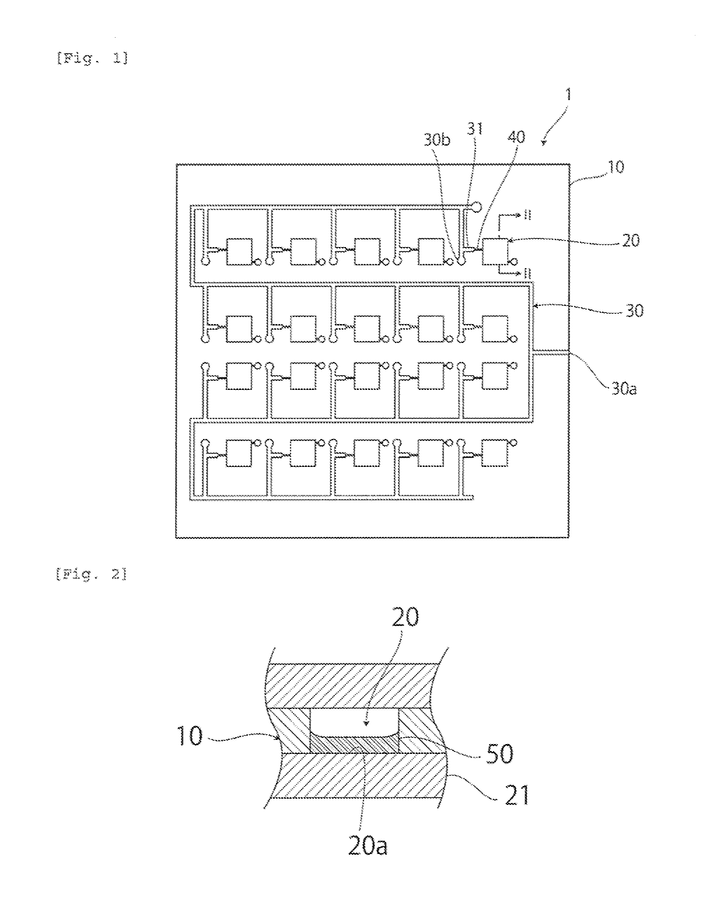

FIG. 1 is a schematic diagram of a nucleic acid amplification reactor of a first embodiment. FIG. 2 is a schematic cross-sectional view of a substrate of the nucleic acid amplification reactor taken along the line II-II in FIG. 1. Referring to FIGS. 1 and 2, the nucleic acid amplification reactor 1 of the first embodiment will be described.

The nucleic acid amplification reactor 1 is a reactor for use in a nucleic acid amplification reaction, such as a PCR method. The nucleic acid amplification reactor 1 is used with a nucleic acid amplification reaction apparatus including a thermal cycler or the like, and a nucleic acid amplification reaction is performed inside the nucleic acid amplification reactor 1.

As shown in FIG. 1, the nucleic acid amplification reactor 1 includes a plurality of reaction chambers 20. As shown in FIG. 2, a thermoplastic hydrogel 50 is applied to each reaction chamber 20. The thermoplastic hydrogel 50 contains a DNA polymerase, a set of oligonucleotide primers, a nucleotide, and a gelator.

The thermoplastic hydrogel 50 causes a phase transition from a gel to a sol when it reaches a gel-sol transition temperature which is a temperature of transition thereof from gel to sol phase. Furthermore, the thermoplastic hydrogel 50 causes a phase transition from a sol to a gel when it reaches a sol-gel transition temperature which is a temperature of transition thereof from sol to gel phase.

The gel-sol transition temperature of the thermoplastic hydrogel 50 is preferably 90 degrees Celsius or below. The sol-gel transition temperature of the thermoplastic hydrogel 50 is preferably 55 degrees Celsius or below. The gel-sol transition temperature and sol-gel transition temperature of the thermoplastic hydrogel 50 can be measured by differential scanning calorimetry (DSC).

The shear elasticity of the thermoplastic hydrogel 50 is preferably about 10³ Pa to about 10⁵ Pa. If the shear elasticity of the thermoplastic hydrogel 50 is about 10³ Pa to about 10⁵ Pa, the applied thermoplastic hydrogel 50 can be allowed to adhere to the nucleic acid amplification reactor 1.

The thermoplastic hydrogel 50 may be a dried product. If the thermoplastic hydrogel 50 is a dried product, its shear elasticity can be changed by adding a fluid, such as a buffer solution, to the dried product of the thermoplastic hydrogel 50.

The thermoplastic hydrogel 50 tends to form a large number of small junction zones when rapidly cooled, while it tends to form a large junction zone when slowly cooled. In the large junction zone, the DNA polymerase, the set of oligonucleotide primers, and the nucleotide dispersed in the thermoplastic hydrogel 50 are likely to cause side reactions. Therefore, if the thermoplastic hydrogel 50 is a dried product, it is desirably a product obtained by drying a thermoplastic hydrogel by rapid freezing.

The gelator contained in the thermoplastic hydrogel 50 is preferably natural polysaccharide, for example. Specific examples of the gelator include agarose, gelatin, carrageenan, gellan gum, xanthan gum, hyaluronic acid, locust bean gum, and polyacrylamide. Of these, the preferred gelator is agarose. A hydrogel of 1% by mass of agarose causes a phase transition to a sol when its temperature rises to approximately 65 degrees Celsius. On the other hand, a hydrosol of 1% by mass of agarose is in a sol phase until approximately 37 degrees Celsius but causes a phase transition to a gel when its temperature drops to approximately 25 degrees Celsius. For example, if agarose is used as a gelator, the thermoplastic hydrogel 50 may have a large hysteresis in terms of the gel-sol transition temperature and the sol-gel transition temperature. If a commonly-used gellatin is used as a gelator, the gel-sol transition temperature of the thermoplastic hydrogel 50 is approximately 26 degrees Celsius. If 2% by mass of k-carrageenan (kappa-carrageenan) is used as a gelator, the gel-sol transition temperature of the thermoplastic hydrogel 50 is approximately 50 degrees Celsius. If 2% by mass of xanthan gum is used as a gelator, the gel-sol transition temperature of the thermoplastic hydrogel 50 is approximately 40 degrees Celsius.

The DNA polymerase is preferably a heat-resistant enzyme DNA polymerase. Specific examples of the DNA polymerase include rTth DNA polymerase.

A set of a forward primer and a reverse primer is appropriately selected as each set of oligonucleotide primers depending upon the nucleic acid sequence which is desired to be amplified. Examples of the nucleotide that can be used include dNTPs (a mixture of four types of deoxyribonucleoside triphosphates (dATP, dCTP, dGTP, and dTTP))

The thermoplastic hydrogel 50 may contain other components necessary for the nucleic acid amplification reaction, such as a magnesium salt. In this embodiment, the thermoplastic hydrogel 50 contains a magnesium salt. An example of the magnesium salt is magnesium chloride (MgCl₂).

If the nucleic acid amplification reactor 1 is used for areal-time PCR method, the thermoplastic hydrogel 50 preferably further contains a reporter reagent. Examples of the reporter reagent include SYBR Green I and TaqMan probe. If the nucleic acid amplification reactor 1 is used for an RT-PCR method, the thermoplastic hydrogel 50 preferably further contains a reverse transcriptase. The reverse transcriptase used is appropriately selected depending upon the type of RNA.

The thermoplastic hydrogel 50 may contain polyvinyl alcohol. Repeatedly cooled and heated polyvinyl alcohol will be gelated at low temperatures, so that it can act as a gelator providing a thermoplastic hydrogel 50. The thermoplastic hydrogel 50 may contain a quality stabilizer, such as a preservative, a chelator or glycerin.

The reaction chamber 20 is composed of a substrate 10. No particular limitation is placed on the material of the substrate 10, provided that it can form a reaction chamber. The substrate 10 can be made from, for example, glass, resin, ceramic, metal or stone. As shown in FIG. 2, the substrate 10 further includes a metallic member 21 provided to extend from the inside wall 20a of the reaction chamber 20 to the outside wall of the nucleic acid amplification reactor 1. Examples of the metal forming the metallic member 21 include aluminum and steel alloys.

In the nucleic acid amplification reactor 1, a sample containing a template DNA and the like is added into the reaction chamber 20 to which the thermoplastic hydrogel 50 is applied. Then, the nucleic acid amplification reactor 1 is heated with a thermal cycler or the like to allow the thermoplastic hydrogel 50 to cause a phase transition to a sol, so that the DNA polymerase, the set of oligonucleotide primers, the nucleotide, and the sample, such as a template DNA, are dispersed in the sol to promote a nucleic acid amplification reaction.

The nucleic acid amplification reactor 1 includes the reaction chambers 20 to each of which is applied a thermoplastic hydrogel 50 containing a DNA polymerase, a set of oligonucleotide primers, and a nucleotide. Therefore, simply by adding a sample, such as a template DNA, into the reaction chamber 20, a nucleic acid amplification reaction can be easily performed. Furthermore, since the DNA polymerase, the set of oligonucleotide primers, and the nucleotide are contained in the thermoplastic hydrogel 50, these components are less likely to react with one another. Thus, even if the nucleic acid amplification reactor 1 is stored for long periods, undesirable side reactions are less likely to occur in the thermoplastic hydrogel 50.

If the nucleic acid amplification reactor 1 further includes a metallic member 21 provided to extend from the inside wall 20a of the reaction chamber 20 to the outside wall of the nucleic acid amplification reactor 1, the temperature control on the nucleic acid amplification reaction can be facilitated.

The nucleic acid amplification reactor 1 can be suitably used not only for the amplification of DNA fragments but also for the detection of a minute amount of RNA in an RT-PCR method. Furthermore, the nucleic acid amplification reactor 1 can be also used for the detection of a minute amount of antigen as part of an immuno-PCR method.

The nucleic acid amplification reactor 1 can employ a hot-start technique using a heat-resistant enzyme DNA polymerase and an anti-DNA polymerase antibody. The hot-start using an antibody exhibits a strong effect on the prevention of undesirable nonspecific reactions. In addition, the hot-start using an antibody allows the antibody to be rapidly deactivated by heat application, so that the reactivation of the enzyme can be expedited. Therefore, the adoption of the hot-start technique can minimize damage to the template RNA and the enzyme due to high temperatures.

The nucleic acid amplification reactor 1 further includes a microchannel 30, weighing parts 31, and passive valves 40. The microchannel 30, the weighing parts 31, and the passive valves 40 are formed in the substrate 10. The weighing parts 31 are connected to the microchannel 30. The weighing parts 13 are provided for the individual reaction chambers 20. The passive valves 40 connect their respective weighing parts 31 to their respective reaction chambers 20.

The term “microchannel” used in the present invention refers to a channel formed in a geometry in which liquid flowing through the microchannel is strongly influenced by surface tension and capillarity to exhibit different behavior from liquid flowing through a channel with a normal size. In short, the term “microchannel” refers to a channel formed in a size that allows liquid flowing therethrough to express a so-called micro effect.

However, what geometry of a channel expresses a micro effect depends upon the physicality of liquid introduced into the channel. For example, if the microchannel has a rectangular cross section, generally, the smaller of the height and width of the cross section of the microchannel is selected to be 5 mm or less, preferably 500 um (micro meter) or less, and more preferably 200 um or less. If the microchannel has a circular cross section, generally, the diameter of the microchannel is selected to be 5 mm or less, preferably 500 um or less, and more preferably 200 um or less.

The microchannel 30 has an opening 30a which opens to the outside of the nucleic acid amplification reactor 1. In the nucleic acid amplification reactor 1, a sample containing a template DNA, a buffer solution and other components is introduced in a microfluidic form into the microchannel 30 through the opening 30a thereof. The sample introduced into the microchannel 30 is fed through the weighing parts 31 to their respective reaction chambers 20.

More specifically, first, the sample is fed to the microchannel 30 and the weighing parts 31. At this point of time, because the passive valves 40 located between their respective weight parts 31 and reaction chambers 20 are formed to be narrow, the sample has not been fed to the reaction chambers 20. Next, a medium immiscible with the sample, such as oil, is introduced through the opening 30a into the microchannel 30 to expel excess sample residing in portions of the microchannel 30 other than the weighing parts 31 through openings 30b connected to the microchannel 30. Thus, a specified amount of weighed sample portion is left in each weighing part 31. Then, when a pressure is applied through the opening 30a to the medium with the openings 30b closed, the sample portions in the weighing parts 31 are fed to their respective reaction chambers 20. The medium immiscible with the sample, such as oil, prevents the contents of the reaction chambers 20 from flowing back during a thermal cycle of a PCR. Air may intervene as a pressure transmission medium for applying a pressure to the above medium.

If the nucleic acid amplification reactor 1 includes the microchannel 30, the weighing parts 31 connected to the microchannel 30 and provided for the individual reaction chambers 20, and the passive valves 40 connecting the weight parts 31 to their respective reaction chambers 20, portions of the sample, such as a template DNA, can be added concurrently and quantitatively into the reaction chambers 20. Thus, a nucleic acid amplification reaction can be more easily performed.

If the nucleic acid amplification reactor 1 includes a plurality of reaction chambers 20, the thermoplastic hydrogel 50 previously applied to each of the plurality of reaction chambers 20 can be of a single type or a combination of types selected from different types of thermoplastic hydrogels different in the type of the set of oligonucleotide primers. Thus, a plurality of different nucleic acid amplification reactions using different sets of oligonucleotide primers can be concurrently performed.

The nucleic acid amplification reactor 1 preferably includes seven or more reaction chambers 20. A thermoplastic hydrogel containing one or more sets of oligonucleotide primers selected from three or more different sets of oligonucleotide primers is applied to each of the seven or more reaction chambers. The set of oligonucleotide primers contained in the thermoplastic hydrogel applied to each of the seven or more reaction chambers is selected according to a recurring pseudo-random binary sequence. In this case, based on Equation (1) below, a column vector C representing the initial concentrations of templates associated with their respective sets of oligonucleotide primers can be determined from a column vector S representing signals observed at the reaction chambers 20.

Using as an example the case where seven reaction chambers 20 and three different sets of oligonucleotide primers are used and a 7-bit M-sequence (maximum length sequence) [1, 1, 1, 0, 0, 1, 0] is selected as a recurring pseudo-random binary sequence, a description is now given of a method for selecting sets of oligonucleotide primers according to the recurring pseudo-random binary sequence.

An M-sequence is a code string having a 2ⁿ-1 digit period generated by an n-bit shift register widely used in, for example, the field of digital communications and feedback. An M-sequence is an example of a recurring pseudo-random binary sequence.

The following matrix is taken as a specific example of a 7×3 matrix M representing whether each of the sets of oligonucleotide primers P0, P1, and P2 associated with their respective templates T0, T1, and T2 is put into each reaction chamber 20.

$$M = {[ \begin{matrix}1 , 1 , 1 , 0 , 0 , 1 , 0 \\ 1 , 0 , 0 , 1 , 0 , 1 , 1 \\ 0 , 1 , 0 , 1 , 1 , 1 , 0\end{matrix} ]}^{T} \qquad [ Math . 1 ]$$

In the above equation, the notation [ ]^{T} indicates a transposition in which rows are swapped with columns. The element M_{i,j} of the matrix M in the i-th row and the j-th column represents in binary-digital form whether the j-th set of oligonucleotide primers is put into the i-th reaction chamber 20. If the element is 1, this means that the set of oligonucleotide primers is put into the reaction chamber. If the element is 0, this means that the set is not put into the reaction chamber. In relation to the elements forming the individual columns, the shift amounts of the recurring pseudo-random binary sequences are 0, 2, and 4. However, the combination of the shift amounts is not limited to this and the shift amounts only have to differ from one column to another.

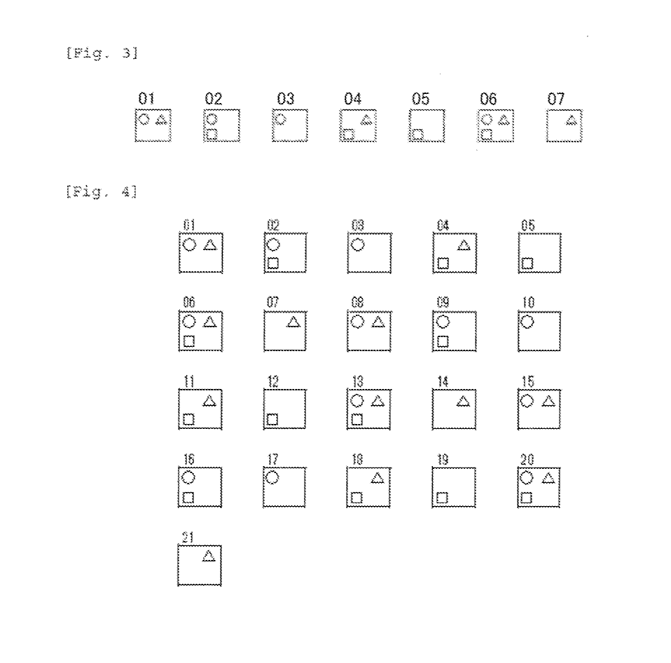

A schematic illustration of this example will be, for example, as shown in FIG. 3. In FIG. 3, the numbered frames represent reaction chambers 20, wherein the circle, triangle, and square show that the thermoplastic hydrogel 50 applied thereto contain P0, P1, and P2, respectively.

If, as another example, twenty-eight reaction chambers 20 and three different sets of oligonucleotide primers are used and three cycles of a 7-bit M-sequence [1, 1, 1, 0, 0, 1, 0, 1, 1, 1, 0, 0, 1, 0, 1, 1, 1, 0, 0, 1, 0] are selected as a recurring pseudo-random binary sequence, the matrix M is as follows:

$$M = {[ \begin{matrix}1 , 1 , 1 , 0 , 0 , 1 , 0 , 1 , 1 , 1 , 0 , 0 , 1 , 0 , 1 , 1 , 1 , 0 , 0 , 1 , 0 \\ 1 , 0 , 0 , 1 , 0 , 1 , 1 , 1 , 0 , 0 , 1 , 0 , 1 , 1 , 1 , 0 , 0 , 1 , 0 , 1 , 1 \\ 0 , 1 , 0 , 1 , 1 , 1 , 0 , 0 , 1 , 0 , 1 , 1 , 1 , 0 , 0 , 1 , 0 , 1 , 1 , 1 , 0\end{matrix} ]}^{T} \qquad [ Math . 2 ]$$

A schematic illustration of this example will be as shown in FIG. 4.

Signals S are obtained as a result of a real-time PCR conducted, using combinations of primer sets arranged according to the matrix M, on an unknown sample containing three or more types of templates in a quantitative ratio represented by a column vector C. If the device function in this case is represented by a matrix A, the relation can be described in the following equation:

[Math. 3]

AMC=S. (1)

In the above equation, C denotes a column vector relating to the initial concentrations of three or more types of templates. If the number of templates is three, the column vector has three elements (c₁, c₂, c₃), they are usually logarithmic scale. Furthermore, S denotes a column vector indicating the magnitudes of signals detected at N reaction chambers. The vector S has N elements (s₁, s₂, s₃, . . . , s_{N}) corresponding to the number of reaction chambers 20.

Next, a description will be given below of how the initial concentrations C of a large number of templates are determined.

Multiplying both sides of Equation (1) shown in Math. 3 by a matrix M* from the left gives the following equation:

[Math. 4]

M*AMC=M*S. (2)

If M* is determined so that a matrix (M*AM) is a regular matrix, an inverse matrix can be obtained from the matrix (M*AM) Thus, the column vector C can be easily obtained from Equation (2). The following matrix is an example of such a matrix M* for the matrix M composed of a single cycle of a 7-bit M-sequence shown in Math. 1.

$${M}^{*} = [ \begin{matrix}1 \text{/} 4 , 1 \text{/} 4 , 1 \text{/} 4 , - 1 \text{/} 3 , - 1 \text{/} 3 , 1 \text{/} 4 , - 1 \text{/} 3 \\ 1 \text{/} 4 , - 1 \text{/} 3 , - 1 \text{/} 3 , 1 \text{/} 4 , - 1 \text{/} 3 , 1 \text{/} 4 , 1 \text{/} 4 \\ - 1 \text{/} 3 , 1 \text{/} 4 , - 1 \text{/} 3 , 1 \text{/} 4 , 1 \text{/} 4 , 1 \text{/} 4 , - 1 \text{/} 3\end{matrix} ] \qquad [ Math . 5 ]$$

This matrix M* can be obtained by replacing each element of the matrix M in the i-th row and j-th column in accordance with the following rules:

[Math. 6]

If M_{i,j}=1,M*_{j,1}=1/(the number of 1s contained in the j-th column of the matrix M); and (1)

If M_{i,j}=0,M*_{i,j}=−1/(the number of 0s contained in the j-th column of the matrix M). (2)

The matrix A which is a device function is a matrix representing device-specific characteristics including not only the relation between signal and initial concentration but also device characteristics, such as lighting bias and sensitivity variations of an image pickup device. This matrix is determined through calibration tests but, for an ideal device, is a unit matrix whose diagonal elements only have a value of 1.

The matrix M*AM is a regular matrix and, particularly for the above ideal device, can be expressed as follows:

$$[ \begin{matrix}1 , - 1 \text{/} 6 , - 1 \text{/} 6 \\ - 1 \text{/} 6 , 1 , - 1 \text{/} 6 \\ - 1 \text{/} 6 , - 1 \text{/} 6 , 1\end{matrix} ] \qquad [ Math . 7 ]$$

Thus, all the quantities in Equation (2) except for the column vector C have been known, so that Equation (2) can be solved for the column vector C. Specifically, from signals S observed at the reaction chambers 20, the initial concentrations C of templates associated with their respective sets of oligonucleotide primers can be determined.

Furthermore, if M*S is calculated assuming that S=[1, 1, 1, . . . , 1]^{T}, it can be confirmed that the values thereof are zero. This shows that in respect of background signals and random noises as based on undesirable side reactions generated before a nucleic acid amplification reaction, their contributions to the calculation for determining the column vector C are strongly canceled.

In the example shown in FIG. 3, four tests for each set of oligonucleotide primers are conducted in a single cycle. With the use of a greater number of reaction chambers than that in a single cycle, the number n of real tests per reagent increases and the error margin decreases in proportion to 1/(square root of n). If tests are conducted in three cycles as in the example shown in FIG. 4, n=12 and the error margin is improved to one fourth of that when n=1. For comparison, assume that different nucleic acid amplification reactions are individually generated for different types of templates in separate reaction chambers 20. If three types of templates are each subjected to four tests and positive and negative control tests are conducted, at least fourteen reaction chambers 20 are required. Alternatively, if three types of templates are each subjected to two tests and positive and negative control tests are conducted, at least eight reaction chambers 20 are required. If, as in this embodiment, the thermoplastic hydrogel contains one or more sets of oligonucleotide primers selected from three or more different sets of oligonucleotide primers and whether each reaction chamber 20 contains a particular set of oligonucleotide primers is determined according to a recurring pseudo-random binary sequence, the required number of reaction chambers 20 can be significantly reduced.

Next, a description will be given of a calibration method of the matrix A.

In the real-time PCR, the rising time of the relative fluorescence intensity (hereinafter referred to as a “Ct value”) of a template vary depending upon the initial concentration of the template. As the initial amount of DNA is greater, the amount of amplification product more rapidly reaches a detectable amount and, therefore, the amplification curve rises in an earlier cycle. Therefore, if the real-time PCR is performed using stepwise diluted standard samples, amplification curves are obtained which are spaced at even intervals in decreasing order of initial DNA amount. When a threshold value is appropriately selected, intersections of the threshold value with the amplification curves, Ct values (threshold cycle), are calculated. Between signals s obtained as Ct values and logarithmic initial DNA concentrations c, there is a linear relationship of c=as+b, therefore, a calibration curve can be formed. In a normal real-time PCR, for a sample having an unknown concentration, the initial template concentration is obtained from the above calibration curve. In this embodiment, however, the calibration curve is not necessary. The calibration of the matrix A is carried out instead.

Ct values observed at N reaction chambers are used as respective values of the elements of the column vector S representing the magnitudes of signals detected at the N reaction chambers. Specifically, S=[s₁, s₂, s₃, . . . , s_{N}]^{T}. If the matrix A serving as a device function is subjected to a first-order approximation, a matrix is obtained of which all of diagonal elements are 1/a, where a corresponds to the slope of the calibration curve in the conventional method. However, if a higher-order band matrix is considered, the calibration can be made with a higher precision. The matrix A can be determined in at least three tests in the case of a second-order approximation and in at least seven tests even in th case of a fourth-order approximation.

Seven tests written in a single matrix is, for example, as follows:

$$D = {[ \begin{matrix}1 , 2 , 3 , 3 , 3 , 4 , 4 \\ 1 , 1 , 1 , 2 , 2 , 3 , 4 \\ 0 , 1 , 1 , 2 , 3 , 4 , 4\end{matrix} ]}^{T} \qquad [ Math . 8 ]$$

The matrix D shows at what quantitative ratio the set of templates T0, T1, and T2 are combined in each test. Specifically, the quantitative ratios are (1, 1, 0) in the first test, (2, 1, 1) in the second test, (3, 1, 1) in the third test, (3, 2, 2) in the fourth test, (3, 2, 3) in the fifth test, (4, 3, 4) in the sixth test, and (4, 4, 4) in the seventh test. In this case, the relation can be expressed in the following Equation (3):

[Math. 9]

AD[c₁,c₂,c₃]^{T}=S. (3)

The matrix A can be determined from Equation (3) by arranging templates having known initial concentrations [c₁, c₂, c₃] in the reaction chambers according to the matrix D and measuring signals S. The matrix D used here is illustrative only and each row of the matrix D is arbitrary within the combinations made by addition and subtraction in each row of the matrix M.

If in a recurring pseudo-random binary sequence the member thereof is represented by m[n], the element by element product of m[n] and m[n−d1] cyclically shifted from m[n] by d1 gives a sequence m[n−d2] cyclically shifted from the original sequence m [n] by d2 In other words, the recurring pseudo-random binary sequence is defined as a sequence having the characteristic of m[n−d2]=m[n]m[n−d1]. A representative example of such a sequence is an M-sequence. Examples of the recurring pseudo-random binary sequence includes, besides the M-sequence, a Gold sequence and other sequences. The sequence for use in determining the arrangement of sets of oligonucleotide primers in the present invention need only be a recurring pseudo-random binary sequence.

The M-sequence is a 1-bit sequence generated from the following linear recurrence formula:

xₙ=xₙ₋ₚ+x_{n-q}(p>q) [Math. 10]

In this linear recurrence formula the value of each term is 0 or 1. The sign “+” represents an exclusive OR (XOR) operation. In other words, the n-th term can be obtained by XORing the n-p-th term and n-q-th term.

The nucleic acid amplification reactor 1 may be provided with a single reaction chamber 20. The shape of the nucleic acid amplification reactor 1 is not limited to that in this embodiment and may be a tubular shape or multiplate shape with none of the microchannel 30, the openings 30a and 30b, the weighing part 31, and the passive valve 40.

### Second Embodiment

The above first embodiment describes the case where the thermoplastic hydrogel 50 contains a magnesium salt. However, the present invention is not limited to the above embodiment. FIG. 6 is a schematic cross-sectional view of a substrate of a nucleic acid amplification reactor of a second embodiment. As shown in FIG. 6, in the second embodiment, the nucleic acid amplification reactor 1 further includes a thermoplastic hydrogel 60 applied to the reaction chamber 20 and containing a magnesium salt. The same type of hydrogel as the thermoplastic hydrogel 50 can be used as the thermoplastic hydrogel 60 containing a magnesium salt

If in the nucleic acid amplification reactor 1 a magnesium salt is contained in the thermoplastic hydrogel 60, undesirable side reactions are less likely to occur because the magnesium salt is less likely to make contact with the DNA polymerase, the set of oligonucleotide primers, and the nucleotide which are contained in the thermoplastic hydrogel 50.

The present invention will be described below in further detail with reference to a specific experimental example. However, the present invention is not limited at all to the following experimental example and appropriate modifications can be made thereto without departing from the gist of the invention.

### Example 1

A PCR reaction liquid (having a total amount of 20 uL (micro liter)) was prepared by mixing the following components (1) to (9) at 55 degrees Celsius to give the following conditions

(1) 14 uL of ultrapure water,

(2) 2 uL of 10×PCR buffer,

(3) 1.2 uL of MgCl₂ aqueous solution (25 mM) (final concentration: 1.5 mM),

(4) 1.6 uL of dNTPs (2.5 mM) (final concentration: 0.2 mM)

(5) 0.2 uL of forward primer (100 pmole) (final concentration: 1 pmole),

(6) 0.2 uL of reverse primer (100 pmole) (final concentration: 1 pmole),

(7) 0.1 uL of rTth DNA polymerase,

(8) 0.5 uL of 1×SYBR Green I, and

(9) 0.2 uL of agarose (Agarose ME manufactured by IWAI CHEMICALS COMPANY LTD.).

A nucleic acid sequence of “CTT CTA ACC GAG GTC GAA ACG TA” and a nucleic acid sequence of “TTG GAC AAA GCG TCT ACG CTG C” were used as a forward primer and a reverse primer, respectively. The target nucleic acid (template) for these oligonucleotide primers was cDNA corresponding to RNA of an MP genome of influenza.

The resultant PCR reaction liquid was dispensed in units of 2.0 uL into a multiplate for PCR and cooled in an atmosphere of 4 degrees Celsius to solidify it. The PCR reaction liquid was gelated on the bottoms of the wells of the multiplate and allowed to adhere thereto. The resultant gel is a thermoplastic hydrogel.

Next, 5 uL of aqueous solution was prepared which contains, as a target nucleic acid serving as a sample, 10 ng of synthesized cDNA corresponding to the MP genome.

Next, the DNA fragments of the sample were amplified by adding 0.5 uL of sample aqueous solution to the wells of the multiplate to which th PCT reaction liquid was applied and repeating a cycle of Operations 1 to 3 described below forty times. As a multiplate to which the PCR reaction liquid was applied, a multiplate 12 hours after the application of the PCR reaction liquid thereto was used.

(Operation 1)

The multiplate is raised in temperature to 95 degrees Celsius and then held at this temperature for 30 seconds to denture the DNA into single-stranded DNAs. At the first temperature rise, the gel is melted and mixed with the sample. The multiplate may be supplementarily vibrated by a piezo vibrator.

(Operation 2)

The mixture is rapidly cooled to about 60 degrees Celsius (which may be slightly different depending upon the oligonucleotide primer used) and then held at this temperature for 30 seconds to anneal the single-chain DNAs obtained in Operation 1 and the oligonucleotide primers.

(Operation 3)

The mixture is raised in temperature again to 72 degrees Celsius and held at this temperature for 10 seconds. At this temperature, no separation of the oligonucleotide primers occurs. This temperature is within the temperature range suitable for activation of the DNA polymerase and is set at about 60 degrees Celsius to 72 degrees Celsius depending upon the purpose of the experiment.

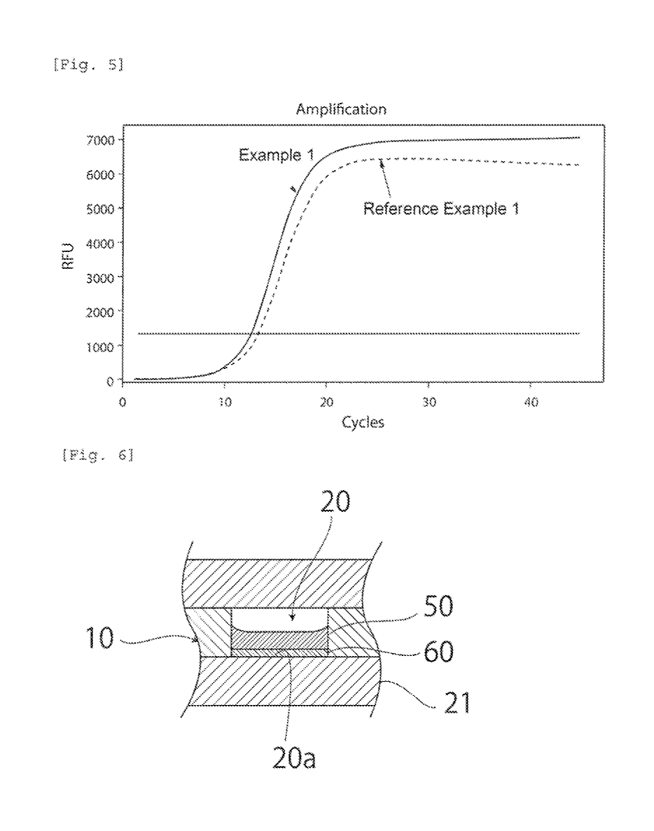

If Operations 2 and 3 are conducted at the same temperature, the cycle is composed of two steps. A graph representing the relation between the amount of DNA fragments and the number of cycles is shown in FIG. 5. In the graph of FIG. 5, the ordinate represents the RFU (relative fluorescent unit) value and the abscissa represents the number of cycles, each composed of Operations 1 to 3.

### Reference Example 1

DNA fragments of a sample were amplified in the same manner as in Example 1 except that instead of agarose the same amount of pure water was used. A graph representing the relation between the amount of DNA fragments and the number of cycles is shown in FIG. 5.

As is apparent from the results of Example 1 and Reference Example 1, also in the case where the PCR reaction liquid containing agarose was used, DNA fragments could be amplified like the case where agarose was not used.

### LIST OF REFERENCE CHARACTERS

1 . . . Nucleic acid amplification reactor
10 . . . Substrate
20 . . . Reaction chamber
20a . . . Inside wall
30 . . . Microchannel
30a, 30b . . . Opening
31 . . . Weighing part
40 . . . Passive valve
50, 60 . . . Thermoplastic hydrogel

1. A nucleic acid amplification reactor comprising:
a reaction chamber to which a thermoplastic hydrogel containing a DNA polymerase, a set of oligonucleotide primers, a nucleotide, and a gelator is applied;
a microchannel;
a weighing part connected to the microchannel and provided for the reaction chamber; and
a passive valve connecting the weighing part and the reaction chamber,
wherein the reaction chamber comprises seven or more reaction chambers,
each of the seven or more reaction chambers includes the thermoplastic hydrogel applied thereto, the thermoplastic hydrogel containing one or more sets of oligonucleotide primers selected from three or more different sets of oligonucleotide primers, and
the set of oligonucleotide primers contained in the thermoplastic hydrogel applied to each of the seven or more reaction chambers is selected according to a recurring pseudo-random binary sequence.

2. The nucleic acid amplification reactor according to claim 1, wherein the gel-sol transition temperature of the thermoplastic hydrogel which is a temperature of transition thereof from gel to sol phase is 90 degrees Celsius or below and the sol-gel transition temperature of the thermoplastic hydrogel which is a temperature of transition thereof from sol to gel phase is 55 degrees Celsius or below.

3. The nucleic acid amplification reactor according to claim 1, wherein the thermoplastic hydrogel further contains a reporter reagent.

4. The nucleic acid amplification reactor according to claim 1, further comprising a thermoplastic hydrogel applied to each of the seven or more reaction chambers and containing a magnesium salt.

5. The nucleic acid amplification reactor according to claim 1, further comprising a metallic member provided to extend from an inside wall of the reaction chamber to an outside wall of the nucleic acid amplification reactor.

6. (canceled)

7. (canceled)

8. (canceled)

9. The nucleic acid amplification reactor according to claim 1, wherein
the weighing part is provided for each of the seven or more reaction chambers.
